(19) United States
(12) Patent Application Publication
(10) Pub. No.: US 2018/0009841 A1
(43) Pub. Date: Jan. 11, 2018

(54) METHOD FOR PREPARING NANOPRECIPITATES OF LOW MOLECULAR WEIGHT PEPTIDE OR PROTEIN

(71) Applicant: Carlina Technologies, Angers (FR)

(72) Inventors: Olivier Meyer, Angers (FR); Thomas Perrier, Saint Barthelemy d'Anjou (FR); Maud Gonnet, Angers (FR)

(73) Assignee: Carlina Technologies, Angers (FR)

(21) Appl. No.: 15/506,446

(22) PCT Filed: Aug. 26, 2015
(86) PCT No.: PCT/EP2015/069546

§ 371 Date: Oct. 2, 2017

(30) Foreign Application Priority Data
Aug. 26, 2014 (EP) 14306318.8

(51) Int. Cl. C07K 1/32; C07K 14/62; A61K 38/00

(52) U.S. Cl. CPC C07K 1/32; C07K 14/62; A61K 38/00

(57) ABSTRACT

The present invention relates to a method for the non-denaturing preparation of peptide or protein nanoprecipitates, or of peptide or protein and metal ion nanocoprecipitales, in which said protein or said peptide has a molecular weight no higher than 20 kDa, preferably no higher than 15 kDa, advantageously no higher than 10 kDa, and more advantageously no higher than 8 kDa. Said method includes a step of preparing a mixture of an aqueous solution of peptides or proteins, a nonsolvent of the peptide or protein, and optionally a water-soluble metal salt. The present invention also relates to a nanoprecipitate that can be obtained by the method according to the invention, as well as to a pharmaceutical composition comprising same, for use in the treatment or prevention of diabetes.

The present invention relates to a method of non-denaturing preparation of peptide or protein nanoprecipitates or of peptide or protein and metal ion, preferably divalent metal ion, nanocoprecipitates, in which the peptides or proteins have a low molecular weight, and to the nanoprecipitates or nanocoprecipitates obtainable by such a method. The present invention also relates to a pharmaceutical composition, particularly a controlled-release pharmaceutical composition, comprising said nanoprecipitates or nanocoprecipitates and to the use of same in the fields of human and animal health.

Peptides and proteins are complex, highly organized structures, highly sensitive to their environment, and can be easily denatured. The denaturation of a peptide or a protein usually leads to a definitive or temporary loss of activity. However, the methods for precipitating peptides or proteins known to date are denaturing because they comprise conditions that are potentially harmful for the structure of the peptides/proteins, which therefore lose their activity, conditions such as high temperatures, rapid stirring or contact with hydrophobic surfaces, aqueous/organic interfaces or detergents.

The nanoprecipitation of peptides or proteins, particularly therapeutic peptides or proteins, makes it possible for example to improve their release profile and their bioavailability in the recipient organism. It also enables the lyophilization of concentrated peptide/protein suspensions and the preparation of pharmaceutical compositions that are highly concentrated in peptides or proteins while having low or no viscosity, comprising the nanoprecipitates obtained in the absence of denaturing aggregation.

There exist in the literature methods of protein nanoprecipitation, such as protein desolvation techniques, which produce protein precipitates of sizes generally greater than a micron. However, these methods usually cause the denaturation of the proteins, or do not make it possible to obtain a protein precipitate without a polymer being necessary in addition to the nonsolvent.

For example, the patent application WO 2009/043874 discloses a method for preparing poloxamer-protein nanoparticles having a mean diameter of 50 to 200 nm and wherein the proteins have a molecular weight preferably higher than 8 kDa and are not denatured. Said nanoparticles are poloxamer-protein complexes; an additional polymer other that the nonsolvent is thus necessary to obtain them.

There thus exists a need to develop a technique for the non-denaturing nanoprecipitation of low molecular weight peptides or proteins, not complexed with another molecule promoting its precipitation or coprecipitated with a metal ion, preferably divalent metal ions. The peptides or proteins are preferably therapeutic peptides or proteins, such as insulin for example.

In the context of the present invention, the inventors discovered a novel method of non-denaturing preparation of nanoprecipitates of peptides or proteins having a molecular weight of 20 kDa or less, preferably of 15 kDa or less, advantageously of 10 kDa or less, more advantageously of 8 kDa or less, making it possible to obtain nanoprecipitates of non-denatured peptides or proteins, or nanocoprecipitates of said non-denatured peptides or proteins and of metal ions, preferably of divalent metal ions, which can be incorporated into a pharmaceutical composition, preferably a controlled-release pharmaceutical composition.

The present invention makes it possible to obtain nanoprecipitates of low molecular weight peptides or proteins, or nanocoprecipitates of low molecular weight peptides or proteins and of metal ions, under mild, non-denaturing conditions. It consists in contacting a solution of low molecular weight peptides or proteins, an organic nonsolvent of peptides or proteins, such as organic diols selected from low molecular weight polyethylene glycols or polyethylene glycol derivatives, particularly PEG 550, and organic diols selected from the group of hexylene glycol, butane-1,4-diol, pentane-1,5-diol, ethohexadiol, 2-methylpentane-2,4-diol(hexylene glycol), 3-cyclopentene-1,2-diol, cis-4-cyclopentene-1,3-diol, trans-1,4-dioxane-2,3-diol, 1,3-dioxane-5,5-dimethanol, (3S,4S)-pyrrolidine-3,4-diol, (3R,4R)-(−)-1-benzyl-3,4-pyrrolidinediol, (3S,4S)-(+)-1-benzyl-3,4-pyrrolidinediol, 3-cyclopentene-1,2-diol, 2-methyl-butane-1,3-diol.

The nanoprecipitation method according to the invention makes it possible to obtain peptide or protein nanoprecipitates or nanocoprecipitates that can be used as a medicinal product for single parenteral administration such that the duration during which the plasma concentration of said peptides or proteins is within the therapeutic window is greater than the duration during which the plasma concentration after a single parenteral administration of the same peptides or proteins not prepared according to the preparation method according to the invention is within the therapeutic window. Thus, the nanoprecipitation according to the invention makes it possible to control the rate of release of the peptides or the proteins, particularly to obtain a sustained-release of the peptide or the protein in a biologically active form for at least 2 days, preferably for 2 days to several months.

The nanoprecipitation according to the invention also enables the preparation of pharmaceutical compositions that are highly concentrated in said peptides or proteins while having low viscosity. The low viscosity of a pharmaceutical composition is particularly useful for the parenteral administration thereof, particularly for the non-intravenous parenteral administration thereof.

In a first aspect, the present invention thus relates to a method of non-denaturing preparation of peptide or protein nanoprecipitates or of peptide or protein and metal ion nanocoprecipitates, having a mean diameter of less than 1 μm, comprising the following steps:
a) preparation of a mixture of an aqueous solution of peptides or proteins, a nonsolvent of the peptides or proteins, and optionally a water-soluble metal salt;
b) gentle stirring of the mixture obtained in step a);
c) solid-liquid separation of the mixture obtained in step b); and
d) optionally, collection of the peptide or protein nanoprecipitates or the peptide or protein and metal ion nanocoprecipitates,
   wherein said peptides or proteins have a molecular weight of 20 kDa or less, preferably of 15 kDa or less, advantageously of 10 kDa or less, more advantageously of 8 kDa or less, and said nonsolvent is selected from polyethylene glycols or polyethylene glycol derivatives having a molecular weight of less than 2 000 Da, advantageously between 200 and 2 000 Da, more advantageously of 550 Da, and organic diols selected from the group of hexylene glycol, butane-1,4-diol, pentane-1,5-diol, ethohexadiol, 2-methylpentane-2,4-diol(hexylene glycol), 3-cyclopentene-1,2-diol, cis-4-cyclopentene-1,3-diol, trans-1,4-dioxane-2,3-diol, 1,3-dioxane-5,5-dimethanol, (3S,4S)-pyrrolidine-3,4-diol, (3R,4R)-(−)-1-benzyl-3,4-pyrrolidinediol, (3S,4S)-(+)-1-benzyl-3,4-pyrrolidinediol, 3-cyclopentene-1,2-diol, 2-methyl-butane-1,3-diol.

In the context of the present invention, by “method of non-denaturing preparation” is meant a method of nanoprecipitation or nanocoprecipitation of peptides or proteins that does not denature said peptides or proteins. The method of nanoprecipitation or nanocoprecipitation according to the invention thus makes it possible to obtain peptides or proteins that retain their normal three-dimensional conformation as well as their integrity and their biological activity. Preferably, the method according to the invention makes it possible to obtain peptide or protein nanoprecipitates or nanocoprecipitates wherein 85%, particularly 90%, more particularly 100%, of the proteins or peptides are not denatured. More preferably, the method according to the invention makes it possible to obtain protein or peptide nanoprecipitates or nanocoprecipitates wherein 100% of the proteins or peptides are not denatured.

In the context of the invention, by “peptide or protein nanoprecipitate” is meant particles having a mean diameter of less than 1 μm, advantageously between 5 nm and 500 nm, more advantageously between 5 nm and 200 nm, even more advantageously between 5 nm and 170 nm, particularly between 5 nm and 150 nm, which are the result of the precipitation of one or more peptides and/or one or more proteins in a nonsolvent. The peptide or protein nanoprecipitates according to the invention consist essentially of the peptide(s) or the protein(s).

According to an advantageous embodiment, the peptide or protein nanoprecipitates according to the invention consist in more than 90%, particularly more than 95%, more particularly more than 99% peptide(s) or protein(s), the remainder being impurities, for example impurities of the nonsolvent used.

Advantageously, the present invention relates to a method of non-denaturing preparation of nanoprecipitates of low molecular weight peptides or proteins or of nanocoprecipitates of low molecular weight peptides or proteins and of metal ions, having a mean diameter of less than 1 μm, advantageously between 5 nm and 500 nm, more advantageously between 5 nm and 200 nm, even more advantageously between 5 nm and 170 nm, particularly between 5 nm and 150 nm.

According to another aspect, the present invention relates to a method of non-denaturing preparation of peptide or protein and metal ion nanocoprecipitates, wherein said peptides or proteins have a molecular weight of 20 kDa or less, preferably of 15 kDa or less, advantageously of 10 kDa or less, more advantageously of 8 kDa or less, and said metal ion is selected from zinc (Zn), manganese (Mn), magnesium (Mg), calcium (Ca), iron (Fe), lithium (Li) and copper (Cu). Preferably, said metal ion is a divalent metal ion selected from zinc (Zn), manganese (Mn), magnesium (Mg), calcium (Ca), iron (Fe) and copper (Cu), preferably zinc (Zn) or manganese (Mn).

By “monovalent metal ion” is meant ions that have a valence of one and that, consequently, can form bonds with another ion or molecule. For example, a monovalent metal anion is an ion that has one additional electron in relation to its elemental state. Likewise, a monovalent metal cation is an ion that has one electron missing in relation to its elemental state.

Likewise, by “divalent metal ion” is meant ions having a valence of two. For example, a divalent metal anion is an ion that has two additional electrons in relation to its elemental state. Likewise, a divalent metal cation is an ion that has two electrons missing in relation to its elemental state.

By “peptide or protein and metal ion nanocoprecipitate”, also called “peptide or protein/metal ion nanocoprecipitate”, is meant the result of coprecipitation of one or more protein(s) or peptide(s) with a metal ion. These nanocoprecipitates thus consist essentially in the peptide(s) or the protein(s), said metal ion and optionally a residue of the nonsolvent used.

By “low molecular weight peptide or protein” is meant proteins or peptides having a molecular weight of 20 kDa or less, preferably of 15 kDa or less, advantageously of 10 kDa or less, more advantageously of 8 kDa or less.

By organic nonsolvent of the peptide or the protein is meant a solvent wherein the protein or the peptide is not soluble, which leads to the formation of a precipitate, preferably a nanoprecipitate.

Included in the “nonsolvents” according to the invention are organic diols selected from polyethylene glycols or polyethylene glycol derivatives having a molecular weight of less than 2 000 Da, advantageously between 200 and 2 000 Da, more advantageously of 550 Da, and organic diols selected from the group of hexylene glycol, butane-1,4-diol, pentane-1,5-diol, ethohexadiol, 2-methylpentane-2,4-diol(hexylene glycol), 3-cyclopentene-1,2-diol, cis-4-cyclopentene-1,3-diol, trans-1,4-dioxane-2,3-diol, 1,3-dioxane-5,5-dim ethanol, (3 S,4S)-pyrrolidine-3,4-diol, (3R,4R)-(−)-1-benzyl-3,4-pyrrolidinediol, (3 S,4S)-(+)-1-benzyl-3,4-pyrrolidinediol, 3-cyclopentene-1,2-diol, 2-methyl-butane-1,3-diol. Preferably, the nonsolvent of the peptide or the protein according to the invention is selected from PEG 550, glycofurol and hexylene glycol ((+)-2-methyl-2,4-pentanediol), preferably from PEG 550 and hexylene glycol. More preferably, the nonsolvent of the peptide or the protein according to the invention is PEG 550.

In the context of the present invention, the expression “low molecular weight polyethylene glycols or polyethylene glycol derivatives” refers to organic diols selected from polyethylene glycols or polyethylene glycol derivatives having a molecular weight of less than 2 000 Da, advantageously between 200 and 2 000 Da, more advantageously of 500 Da, and hexylene glycol. The low molecular weight polyethylene glycols or polyethylene glycol derivatives according to the invention are selected from PEG 100, PEG 200, PEG 300, PEG 400, PEG 550, PEG 600, PEG 900, PEG 1000, PEG 2000, glycofurol. Preferably, the low molecular weight polyethylene glycols or polyethylene glycol derivatives according to the invention are selected from PEG 550 and glycofurol, preferably the low molecular weight polyethylene glycol according to the invention is PEG 550.

In the context of the present invention, the nonsolvent of the peptide or the protein is used in a sufficient quantity to precipitate said peptides or proteins, or to nanocoprecipitate said peptides or proteins and the metal ion, in the form of nanoparticles having a mean diameter of less than 1 μm. The peptide or protein solution/peptide or protein nonsolvent volume ratio can be determined by methods known to the person skilled in the art. The nanoprecipitation or nanocoprecipitation yield depends particularly on the quantity of peptides or proteins to nanoprecipitate or nanocoprecipitate. Indeed, as shown in FIG. 1, the smaller the quantity of peptides or proteins to nanoprecipitate or nanocoprecipitate, the easier the nanoprecipitation or nanocoprecipitation and the greater the yield. An increase in the quantity of peptides or proteins to nanoprecipitate or nanocoprecipitate can lead to a decrease in the yield.

Preferably, the present invention relates to a method of non-denaturing preparation of therapeutic peptide or protein nanoprecipitates or of therapeutic peptide or protein/metal ion nanocoprecipitates.

By “therapeutic peptide or protein” is meant any peptide or any protein the administration of which enables the treatment of one or more pathologies. Among therapeutic peptides or proteins, mention may be made of enzymes, cytokines, growth factors, hormones, antibodies and coagulation factors, as well as diagnostic agents, particularly peptide hormones such as insulins and derivatives, glucagon and analogues (glucagon-like peptides), growth hormones, somatostatins, vasopressins, calcitonins, LHRH (luteinizing-hormone-releasing-hormone) agonists and antagonists, ACTH (adrenocorticotropic hormone), ACTH-synergistic peptides, somatotropic hormone, luteotropic hormone, thyrotropic hormone, follicle stimulating hormone, interstitial cell stimulating hormone (ICSH), thyroliberin, corticoliberin, somatoliberin, luteoliberin, prolactin inhibiting factor, tyrocidine A, penicillins, gramicidins, oxytocin, vaccine peptides, and natural and synthetic derivatives thereof or fragments thereof. Advantageously, the present invention relates to a method of non-denaturing preparation of nanoprecipitates of peptides or proteins selected from human insulin, growth hormone, glucagon, peptide hormones or a therapeutically effective derivative or fragment thereof. More advantageously, the present invention relates to a method of non-denaturing preparation of nanoprecipitates of human insulin or a therapeutically effective derivative or fragment thereof or of a nanocoprecipitate of human insulin or a therapeutically effective derivative or fragment thereof and of metal ions, preferably a human insulin/zinc nanocoprecipitate.

The expression “therapeutically effective derivative” of a peptide or a protein refers to peptides or proteins wherein one or more amino acid residues have been substituted by other amino acid residues and/or wherein one or more amino acid residues of the peptide or the protein have been removed and/or wherein one or more amino acid residues have been added to the peptide or the protein. In particular, the expression “therapeutically effective derivatives” of a peptide or a protein refers to PEGylated, glycosylated, acetylated, phosphorylated derivatives, cyclic derivatives, derivatives coupled to one or more natural or synthetic lipids, to one or more aptamers, to one or more peptide sequences, to one or more nucleic acids, RNA or DNA, or proteins or peptides coupled to a scaffold-type structure having a characterized chemical structure such as dendrimers and multivalent structures. Advantageously, the expression “therapeutically effective derivatives” of a peptide or a protein refers to PEGylated, glycosylated and acetylated peptides or proteins.

By “therapeutically effective fragment” of a peptide or a protein is meant a part of the polypeptide, the peptide or the protein that is shorter than the length of the peptide or the protein in the natural state and that has an effective therapeutic activity. For example, by “therapeutically effective fragment of insulin” is meant a part of the insulin polypeptide that is shorter than the length of the insulin protein in the natural state and that has an effective therapeutic activity, particularly against diabetes.

According to the present invention, the nanoprecipitation of peptides or proteins or the nanocoprecipitation of peptides or proteins and of metal ions occurs over a wide temperature range, particularly at a temperature between −20 and 37° C., more particularly at a temperature between 0 and 20° C. This temperature range makes it possible to limit any risk of denaturation of the peptides or proteins.

In a preferred embodiment, the gentle stirring is carried out according to methods known to the person skilled in the art, particularly by mechanical or magnetic stirring, more particularly by stirring at 50 rpm or lower. The method according to the invention is thus carried out under mild, non-denaturing conditions, involving very low shear forces (Reynolds number below 2 000).

In another preferred embodiment, step b) of the method according to the invention is followed of a step during which the mixture is cooled, advantageously in an ice bath, for 1 minute to 60 minutes at a temperature between 0° C. and 10° C.

In yet another preferred embodiment, the solid/liquid separation of step c) of the method according to the invention is a centrifugation, particularly in a range of centrifugal force between about 500 and 50 000 G. Likewise, step d) of collecting the nanoprecipitates is carried out by membrane filtration or tangential filtration.

The nanoprecipitation can optionally be carried out in the presence of water-soluble metal salt, such as ZnCl₂, MgCl₂, CaCl₂, MnCl₂, FeCl₂, CuSO₄ or LiCl. Preferably, the water-soluble metal salt is zinc chloride (ZnCl₂) or manganese chloride (MnCl₂). This is referred to as nanocoprecipitation according to the invention.

Advantageously, the use of a water-soluble salt in combination with a nonsolvent of the peptides or proteins promotes and/or improves the precipitation of the peptides or proteins, by forming metal ion/protein (or peptide) complexes. The salt makes it possible particularly to obtain better precipitation yields while preserving the activity of the peptides or proteins after precipitation and re-dissolution.

The salt concentration of the aqueous solution can vary in a broad interval. For a given quantity of peptides or proteins, at a fixed pH of the solution, a fixed temperature and a fixed water/water-miscible nonsolvent volume ratio, the person skilled in the art can determine a suitable minimum salt concentration by routine experimentation, typically by addition of increasing quantities of salt until precipitation of the peptides or proteins is observed. However, when the nanoprecipitation is carried out in the presence of water-soluble salt, the nanocoprecipitation yield depends on the quantity of peptides or proteins and on the salt concentration. As shown in FIGS. 2, 3, 4 and 5, when the quantity of peptides or proteins is small, the nanocoprecipitation yield depends strongly on the salt concentration and it increases with the decrease in the salt concentration.

Thus, the method according to the invention can be characterized in that said mixture of step a) also comprises at least one water-soluble metal salt selected from ZnCl₂, MgCl₂, CaCl₂, MnCl₂, FeCl₂, LiCl and CuSO₄, advantageously ZnCl₂ or MnCl₂.

The invention further relates to a protein or peptide nanoprecipitate or a peptide or protein and metal ion nanocoprecipitate, wherein said peptides or proteins have a molecular weight of 20 kDa or less, preferably of 15 kDa or less, advantageously of 10 kDa or less, more advantageously of 8 kDa or less, obtainable by the method according to the invention.

Preferably, the nanoprecipitates or nanocoprecipitates according to the invention consist in more than 85%, particularly more than 90%, more particularly 100% non-denatured proteins or peptides.

Preferably, the nanoprecipitates or nanocoprecipitates according to the invention have a mean diameter of less than 1 μm, advantageously between 5 nm and 500 nm, more advantageously between 5 nm and 200 nm, even more advantageously between 5 nm and 170 nm, particularly between 5 nm and 150 nm. They are collected, in particular, to be incorporated into a suitable pharmaceutical preparation.

More particularly, the peptide or protein nanoprecipitates or nanocoprecipitates according to the invention can be used as a sustained-release medicinal product for controlling the delivery of said peptides or proteins in vivo.

In another aspect, the invention relates to peptide or protein nanoprecipitates or peptide or protein and metal ion nanocoprecipitates according to the invention, for use as a medicinal product.

The invention also relates to peptide or protein nanoprecipitates or peptide or protein and metal ion nanocoprecipitates according to the invention, for use as a medicinal product for single parenteral administration, particularly via non-intravenous parenteral route, such that the duration during which the plasma concentration of said peptides or proteins is within the therapeutic window is greater than the duration during which the blood concentration after a single parenteral administration, particularly via non-intravenous parenteral route, of the same peptides or proteins not prepared according to the preparation method according to the invention is within the therapeutic window.

In the context of the present invention, by “therapeutic window” of an active ingredient is meant all the plasma concentrations of said active ingredient between the minimum therapeutic plasma concentration of said active ingredient, i.e. the minimum plasma concentration at which the active ingredient has an effective therapeutic activity, and the maximum therapeutic plasma concentration of said active ingredient, i.e. the maximum plasma concentration of said active ingredient that can be reached without leading to troublesome side effects.

In particular, the invention relates to peptide or protein nanoprecipitates or peptide or protein and metal ion nanocoprecipitates according to the invention, for use as a medicinal product for single parenteral administration, particularly via non-intravenous parenteral route, such that the duration during which the plasma concentration of said peptides or proteins is within the therapeutic window is greater than 2 days, preferably greater than 7 days, preferably greater than 21 days, more preferably greater than 30 days.

More particularly, the invention relates to peptide or protein nanoprecipitates or peptide or protein and metal ion nanocoprecipitates according to the invention, for use as a medicinal product for single parenteral administration, particularly via non-intravenous parenteral route, such that the duration during which the plasma concentration of said peptides or proteins is within the therapeutic window is greater than 21 days, more preferably greater than 30 days.

In another aspect, the invention relates to peptide or protein nanoprecipitates or peptide or protein and metal ion nanocoprecipitates according to the invention, for use as a medicinal product for single parenteral administration, particularly via non-intravenous parenteral route, such that the duration during which the plasma concentration of said peptides or proteins is within the therapeutic window is 2 days longer, preferably 7 days longer, more preferably 21 days longer, even more preferably 30 days longer, than the duration during which the plasma concentration after a single parenteral administration, particularly via non-intravenous parenteral route, of the same peptides or proteins not prepared according to the preparation method according to the invention is within the therapeutic window.

In particular, the invention relates to peptide or protein nanoprecipitates or peptide or protein and metal ion nanocoprecipitates according to the invention, for use as a medicinal product for single parenteral administration, particularly via non-intravenous parenteral route, such that the duration during which the plasma concentration of said peptides or proteins is within the therapeutic window is 21 days longer, preferably 30 days longer, than the duration during which the plasma concentration after a single parenteral administration, particularly via non-intravenous parenteral route, of the same peptides or proteins not prepared according to the preparation method according to the invention is within the therapeutic window.

In still another aspect, the invention relates to pure peptide or protein nanoprecipitates or peptide or protein and metal ion nanocoprecipitates according to the invention, for use as a medicinal product for single parenteral administration, particularly via non-intravenous parenteral route, between Tₘₐₓ and T_{max+2 days}, preferably between Tₘₐₓ and T_{max+7 days}, more preferably between Tₘₐₓ and T_{max+21 days}, even more preferably between Tₘₐₓ and T_{max+30 days}.

More particularly, after the single parenteral administration, particularly via non-intravenous parenteral route, of nanoprecipitates or nanocoprecipitates according to the invention, the plasma peptide or protein concentration is preferably greater than 90%, and more preferably greater than 98%, of the maximum plasma concentration (Cₘₐₓ) value between Tₘₐₓ and T_{max+2 days}, preferably between Tₘₐₓ and T_{max+7 days}, more preferably between Tₘₐₓ and T_{max+21 days}, even more preferably between Tₘₐₓ and T_{max+30 days}.

The term “maximum plasma concentration (Cₘₐₓ)” refers to the peak concentration representing the point where the plasma concentration is highest in the whole kinetics. This peak is reached when the quantities absorbed and eliminated are equal; it is measured after administration of a single dose.

“Tₘₐₓ” is the value of the time necessary to reach the maximum plasma concentration. This value is indicative of the absorption rate of a pharmaceutically active substance.

“T_{max+x days}” corresponds to the time necessary to reach the maximum plasma concentration Tₘₐₓ to which x days are added, x corresponding to an integer, particularly x is equal to 2, 7, 21 or 30. For example, “T_{max+2 days}” corresponds to the value of the time Tₘₐₓ to which 2 days are added.

In a preferred aspect of the present invention, the inventors adjusted the time during which the plasma concentration must be maintained greater than at least 85% of the Cₘₐₓ, value as a function of the active ingredient used. For example and in a nonlimiting manner, pure insulin nanoprecipitates or insulin/zinc nanocoprecipitates according to the invention can be used as an insulin-releasing medicinal product administered only once via parenteral route, particularly via non-intravenous parenteral route, such that the plasma insulin concentration is greater than 85%, preferably greater than 90%, more preferably greater than 98%, of the Cₘₐₓ value between Tₘₐₓ and T_{max+2 days}, preferably between Tₘₐₓ and T_{max+5 days}, particularly between Tₘₐₓ and T_{max+7 days}. Likewise, pure growth hormone nanoprecipitates or growth hormone/metal ion nanocoprecipitates according to the invention are used as a growth hormone-releasing medicinal product administered only once via parenteral route, particularly via non-intravenous parenteral route, such that the plasma growth hormone concentration is greater than 85%, preferably greater than 90%, more preferably greater than 98%, of the Cₘₐₓ value between Tₘₐₓ and T_{max+21 dys}, preferably between Tₘₐₓ and T_{max+30 days}.

In a particular embodiment, the nanoprecipitates or nanocoprecipitates according to the invention enable the sustained-release of said peptides or proteins in vivo, for at least 2 days, advantageously at least 5 days, more advantageously at least 30 days, even more advantageously for 90 days. In an advantageously preferred manner, the nanoprecipitates or nanocoprecipitates according to the invention enable the sustained-release of said peptides or proteins in vive for at least 7 days, advantageously for at least 21 days, more advantageously for 30 days.

The nanoprecipitates or nanocoprecipitates according to the invention are compatible with the use of any system or any polymer matrix making it possible to release the protein or the peptide in a controlled manner over time. Thus, in a preferred embodiment, the nanoprecipitates or nanocoprecipitates according to the invention are incorporated into polymer matrices also enabling their sustained-release in vivo.

In the context of the present invention, by “polymer matrix” is meant a polymer network, optionally three-dimensional when the polymers are crosslinked, such as a gel. Preferably, the polymer matrices comprise in particular natural or synthetic biocompatible polymers selected from polymers based on glycolic acid, on lactic acid, on pluronic acid, on polyethylene glycol, on polysaccharides such as hyaluronic acid, on polypeptides, PEO-PPO (poly(ethylene oxide)-poly(propylene oxide)) triblock copolymers, based on isopropylacrylamide, lipid matrices, dendrimers or a mixture thereof. Preferably, the polymer matrix is biodegradable.

More particularly, the polymer matrix is a gel, particularly an injectable gel, more particularly a thermosensitive gel or a lipid matrix, even more particularly a hyaluronic acid gel.

By “nanoprecipitates or nanocoprecipitates incorporated into polymer matrices” is meant nanoprecipitates or nanocoprecipitates according to the invention that are encapsulated, dispersed, grafted or crosslinked in the polymer matrix.

The present invention also relates to a pharmaceutical composition comprising nanoprecipitates or nanocoprecipitates according to the invention, and advantageously a pharmaceutically acceptable excipient. Advantageously, the pharmaceutical composition according to the invention comprises the nanoprecipitates or nanocoprecipitates according to the invention incorporated into polymer matrices.

According to an advantageous embodiment, the pharmaceutical composition according to the invention is a sustained-release pharmaceutical composition. More particularly, the composition according to the invention releases the peptides or proteins in a prolonged manner for more than 2 days, advantageously more than 5 days, more advantageously more than 30 days, even more advantageously more than 90 days.

The pharmaceutical composition is particularly suited for administration via parenteral, preferably non-intravenous, oral, sublingual, nasal, transdermal, topical, rectal, ocular and mucosal route, preferably via non-intravenous parenteral route. In an advantageously preferred manner, the nanoprecipitates or nanocoprecipitates according to the invention enable the sustained-release of the peptides or proteins in vivo for 7 days, advantageously for 21 days, more advantageously for 30 days.

In a preferred embodiment, the pharmaceutical composition according to the invention releases the proteins or peptides according to the invention after a single parenteral administration, particularly via non-intravenous parenteral route, such that the duration during which the plasma concentration of said peptides or proteins is within the therapeutic window is greater than the duration during which the plasma concentration after a single parenteral administration, particularly via non-intravenous parenteral route, of the same peptides or proteins not prepared according to the preparation method according to the invention is within the therapeutic window.

In particular, the pharmaceutical composition according to the invention releases the proteins or peptides according to the invention after a single parenteral administration, particularly via non-intravenous parenteral route, such that the duration during which the plasma concentration of said peptides or proteins is within the therapeutic window is greater than 2 days, preferably greater than 7 days, preferably greater than 21 days, more preferably greater than 30 days.

In particular, the pharmaceutical composition according to the invention releases the proteins or peptides according to the invention after a single parenteral administration, particularly via non-intravenous parenteral route, such that the duration during which the plasma concentration of said peptides or proteins is within the therapeutic window is 2 days longer, preferably 7 days longer, more preferably 21 days longer, even more preferably 30 days longer, than the duration during which the plasma concentration after a single parenteral administration, particularly via non-intravenous parenteral route, of the same peptides or proteins not prepared according to the preparation method according to the invention is within the therapeutic window.

In another aspect, the pharmaceutical composition according to the invention releases the proteins or peptides according to the invention after a single parenteral administration, particularly via non-intravenous parenteral route, such that the plasma peptide or protein concentration is greater than 85%, preferably greater than 90%, more preferably greater than 98%, of the Cₘₐₓ value between Tₘₐₓ and T_{max+2 days}, preferably between Tₘₐₓ and T_{max+7 days}, preferably between Tₘₐₓ and T_{max+21 days}, even more preferably between Tₘₐₓ and T_{max+30 days}.

Advantageously, the present invention relates to a pharmaceutical composition comprising nanoprecipitates or nanocoprecipitates according to the invention of peptides or proteins selected from human insulin, growth hormone, glucagon, peptide hormones or a therapeutically effective derivative or fragment thereof. More advantageously, the present invention relates to a pharmaceutical composition comprising nanoprecipitates according to the invention of human insulin or a therapeutically effective derivative or fragment thereof or nanocoprecipitates according to the invention of human insulin or a therapeutically effective derivative or fragment thereof and of metal ions, preferably human insulin/zinc nanocoprecipitates.

More particularly, the compositions suited to topical administration include: creams, emulsions, milks, ointments, lotions, oils, aqueous or hydroalcoholic or glycolic solutions, powders, patches, sprays, sticks or any other product for external application. The preparations suited to oral administration can appear in the form of capsules, particularly soft capsules, particularly of gelatin or plants, powder, tablets, particularly to be swallowed or crunched, chewable or effervescent. They can also appear in liquid, emulsion, cream, paste, powder, gel or suspension form. Finally, the compositions suited to administration via non-intravenous parenteral route, i.e. by means of a subcutaneous, intradermal or intramuscular injection, appear particularly in suspension, injectable solution or implant form. The compositions according to the invention can also be combined with medical devices, such as stents.

The modes of administration, dosing schedules and optimal galenic forms of the pharmaceutical composition according to the invention can be determined according to the criteria generally taken into account in the establishment of a pharmaceutical treatment suited to a subject such as, for example, the patient's age or body weight, the seriousness of his general condition, the tolerance to the treatment, the noted side effects.

The pharmaceutically acceptable excipient is known to the person skilled in the art and is selected according to the mode of administration of the pharmaceutical composition. Thus, the pharmaceutically acceptable excipient can be selected from the group consisting of diluents, dispersants, wetting agents, binders, disintegrants, dyes, lubricants, solubilizers, absorption promoters, film-forming agents, gelling agents, and mixtures thereof.

By way of example, a solid composition in tablet form comprises a pharmaceutical carrier such as gelatin, starch, lactose, magnesium stearate, talc, gum arabic or analogues; and optionally a coating of sucrose or of other suitable materials. Likewise, a capsule preparation comprises in particular a thinner and soft or hard capsules. A preparation in syrup or elixir form can contain the active ingredient jointly with a sweetener, an antiseptic, as well as a flavoring agent and a suitable dye. The water-dispersible powders or granules can contain the active ingredient mixed with dispersants or wetting agents, or suspending agents, as well as flavor enhancers or sweeteners. For rectal administration, the suppositories are prepared with binders that melt at rectal temperature, for example cocoa butter or polyethylene glycols. Finally, for parenteral, intranasal or intraocular administration, one uses aqueous suspensions, isotonic saline solutions or sterile injectable solutions that contain pharmacologically compatible dispersants and/or wetting agents.

Advantageously, the pharmaceutical composition according to the invention comprises the nanoprecipitates or nanocoprecipitates according to the invention, preferably of insulin or a therapeutically effective derivative or fragment thereof, or nanocoprecipitates of insulin and of metal ions, preferably of insulin and of zinc, incorporated into a polymer matrix, which is a stable sustained-release system sufficiently fluid to be easily injected and sterilized. It comprises in particular polymers selected from polymers based on glycolic acid, on lactic acid, on pluronic acid, on polyethylene glycol, on polysaccharides such as hyaluronic acid, on polypeptides, PEO-PPO (poly(ethylene oxide)-poly(propylene oxide)) triblock copolymers, based on isopropylacrylamide, lipid matrices, dendrimers, or a mixture thereof. More particularly, the nanoprecipitates according to the invention are formulated within a hyaluronic acid gel.

In a particular aspect, the invention further relates to the sustained-release pharmaceutical composition according to the invention, for use as a medicinal product.

The present invention also relates to nanoprecipitates or nanocoprecipitates according to the invention or a pharmaceutical composition according to the invention, for use in the fields of human and animal health, particularly in oncology, tissue and bone degeneration, ophthalmology, endocrinology (including type I and type II diabetes), cardiovascular disease, dermatology, dermocosmetology, infectiology, parasitology, cardiology, immunology, hepatology, hematology, gastrology, enterology, rheumatology, wounds, pneumology, gynecology, otorhinolaryngology, diagnostic products.

Moreover, the present invention also relates to the use of nanoprecipitates or nanocoprecipitates according to the invention or a pharmaceutical composition according to the invention, for the preparation of a medicinal product, particularly intended for the treatment and/or prevention of a disease in the fields of human and animal health, particularly in oncology, tissue and bone degeneration, ophthalmology, endocrinology (including type I and type II diabetes), cardiovascular disease, dermatology, dermocosmetology, infectiology, parasitology, cardiology, immunology, hepatology, hematology, gastrology, enterology, rheumatology, wounds, pneumology, gynecology, otorhinolaryngology, diagnostic products.

The invention also relates to a method for treating and/or preventing a disease in the fields of human and animal health, particularly in oncology, tissue and bone degeneration, ophthalmology, endocrinology (including type I and type II diabetes), cardiovascular disease, dermatology, dermocosmetology, infectiology, parasitology, cardiology, immunology, hepatology, hematology, gastrology, enterology, rheumatology, wounds, pneumology, gynecology, otorhinolaryngology, diagnostic products, in a subject in need thereof, comprising the administration to the subject of a therapeutically effective quantity of nanoprecipitates or nanocoprecipitates according to the invention or of a pharmaceutical composition according to the invention.

More particularly, the present invention relates to nanoprecipitates of insulin or a therapeutically effective derivative or fragment thereof or nanocoprecipitates of insulin and of metal ions, preferably of insulin and of zinc, according to the invention or a pharmaceutical composition according to the invention, for use in the treatment and/or prevention of diabetes.

In an advantageously preferred manner, the insulin nanoprecipitates or insulin/metal ion nanocoprecipitates according to the invention enable the sustained-release of insulin after a single parenteral administration, particularly via non-intravenous parenteral route, such that the duration during which the plasma insulin concentration is within the therapeutic window is greater than the duration during which the plasma concentration after a single parenteral administration of insulin not prepared according to the preparation method according to the invention is within the therapeutic window.

In particular, the insulin nanoprecipitates or insulin/metal ion nanocoprecipitates according to the invention enable the sustained-release of insulin after a single parenteral administration, particularly via non-intravenous parenteral route, such that the duration during which the plasma concentration of said peptides or proteins is within the therapeutic window is greater than 2 days, preferably greater than 7 days, more preferably greater than 21 days, even more preferably greater than 30 days.

In particular, the insulin nanoprecipitates or insulin/metal ion nanocoprecipitates according to the invention enable the sustained-release of insulin after a single parenteral administration, particularly via non-intravenous parenteral route, such that the duration during which the plasma concentration of said peptides or proteins is within the therapeutic window is 2 days longer, preferably 7 days longer, more preferably 21 days longer, even more preferably 30 days longer, than the duration during which the plasma concentration after a single parenteral administration, particularly via non-intravenous parenteral route, of the same peptides or proteins not prepared according to the preparation method according to the invention is within the therapeutic window.

In another aspect, the insulin nanoprecipitates or insulin/metal ion nanocoprecipitates according to the invention enable the sustained-release of insulin after a single parenteral administration, particularly via non-intravenous parenteral route, such that the plasma insulin concentration is greater than 85%, preferably greater than 90%, more preferably greater than 98%, of Cₘₐₓ between Tₘₐₓ and T_{max+2 days}, advantageously between Tₘₐₓ and T_{max+5 days}, even more advantageously between Tₘₐₓ and T_{max+7 days}.

In a preferred embodiment, the present invention relates to a sustained-release pharmaceutical composition of nanoprecipitates of insulin or a therapeutically effective derivative or fragment thereof or of nanocoprecipitates of insulin and of metal ions, preferably of insulin and of zinc, according to the invention, for use in the treatment and/or prevention of diabetes. Preferably, the pharmaceutical composition according to the invention enables a sustained-release of insulin in vivo of more than 2 days, advantageously of more than 5 days, more advantageously of more than 30 days, even more advantageously for 90 days.

Advantageously, the pharmaceutical composition according to the invention enables the sustained-release after a single parenteral administration, particularly via non-intravenous parenteral route, of insulin or a therapeutically effective derivative or fragment thereof or of insulin/metal ion, preferably insulin/zinc, complexes according to the invention, such that the duration during which the plasma insulin concentration is within the therapeutic window is greater than the duration during which the plasma concentration after a single parenteral administration of insulin not prepared according to the preparation method according to the invention is within the therapeutic window.

In particular, the pharmaceutical composition according to the invention enables the sustained-release after a single parenteral administration, particularly via non-intravenous parenteral route, of insulin or a therapeutically effective derivative or fragment thereof or of insulin/metal ion, preferably insulin/zinc, complexes according to the invention, such that the duration during which the plasma concentration of said peptides or proteins is within the therapeutic window is greater than 2 days, preferably greater than 7 days, preferably greater than 21 days, more preferably greater than 30 days.

In particular, the pharmaceutical composition according to the invention enables the sustained-release after a single parenteral administration, particularly via non-intravenous parenteral route, of insulin or a therapeutically effective derivative or fragment thereof or of insulin/metal ion, preferably insulin/zinc, complexes according to the invention, such that the duration during which the plasma concentration of said peptides or proteins is within the therapeutic window is 2 days longer, preferably 7 days longer, more preferably 21 days longer, even more preferably 30 days longer, than the duration during which the plasma concentration after a single parenteral administration, particularly via non-intravenous parenteral route, of the same peptides or proteins not prepared according to the preparation method according to the invention is within the therapeutic window.

In another aspect, the pharmaceutical composition according to the invention enables the sustained-release after a single parenteral administration, particularly via non-intravenous parenteral route, of insulin or a therapeutically effective derivative or fragment thereof or of insulin/metal ion, preferably insulin/zinc, complexes according to the invention, such that the plasma insulin concentration is greater than 85%, preferably greater than 90%, more preferably greater than 98%, of Cₘₐₓ between Tₘₐₓ and T_{max+2 days}, advantageously between Tₘₐₓ and T_{max+5 days}, even more advantageously between Tₘₐₓ and T_{max+7 days},

In the context of the present invention, by “diabetes” is meant an incurable chronic disease caused by a deficiency or a defective use of insulin leading to excess blood sugar. Two types of diabetes exist; type I diabetes is characterized by the complete absence of insulin production, type 2 diabetes (non-insulin dependent diabetes or NIDD) is a glucose metabolism disorder characterized by an increased glucose level in the blood, hyperglycemia.

The present invention also relates to the use of nanoprecipitates of insulin or a therapeutically effective derivative or fragment thereof or of insulin/metal ion, preferably insulin/zinc, complexes according to the invention or of a pharmaceutical composition according to the invention, for the preparation of a medicinal product intended for the treatment and/or prevention of diabetes.

Moreover, the invention relates to a method for treating and/or preventing diabetes, in a subject in need thereof, comprising the administration to the subject of a therapeutically effective quantity of a nanoprecipitate or nanocoprecipitate according to the invention or of a pharmaceutical composition according to the invention.

### DESCRIPTION OF THE FIGURES

FIG. 1. Insulin nanoprecipitation yield as a function of insulin mass used.

FIG. 2. Insulin nanocoprecipitation yield as a function of insulin mass, in the presence of manganese chloride, MnCl₂ (condition (1) 0.12 mg of MnCl₂; condition (2) 0.25 mg of MnCl₂).

FIG. 3. Insulin nanocoprecipitation yield as a function of insulin mass, in the presence of calcium chloride, CaCl₂ (condition (1) 0.12 mg of CaCl₂; condition (2) 0.25 mg of CaCl₂).

FIG. 4. Insulin nanocoprecipitation yield as a function of insulin mass, in the presence of zinc chloride, ZnCl₂ (condition (1) 0.12 mg of ZnCl₂; condition (2) 0.25 mg of ZnCl₂).

FIG. 5. Influence of zinc chloride concentration on insulin nanoprecipitation yield.

FIG. 6. Influence of temperature on insulin nanoprecipitation or nanocoprecipitation yield with 0.12 mg of ZnCl₂.

FIG. 7. Optical microscopy photographs of polymer microspheres loaded with insulin nanoprecipitates.

FIG. 8. Profile of insulin release from insulin nanoprecipitates with glycofurol (♦) or PEG 550 (▪) according to the invention, from a 1% hyaluronic acid gel.

FIG. 9. Profile of insulin release from insulin nanoprecipitates with glycofurol (♦) or PEG 550 (▪) according to the invention, from a 2% hyaluronic acid gel.

FIG. 10. Profile of insulin release from an insulin and metal ion nanocoprecipitate according to the invention prepared with glycofurol and ZnCl₂(), glycofurol and MnCl₂ (♦), PEG 550 and ZnCl₂ (∘) or PEG 550 and MnCl₂ (⋄), within a hyaluronic acid gel in comparison with the release profile of insulin in native form from a hyaluronic acid gel (▪).

FIG. 11. Insulin release profile as a function of the presence or absence of a step of re-suspension of the nanoprecipitates. This suspension step is carried out before dispersion of the insulin nanoprecipitates within the hyaluronic acid matrix. The condition without prior dilution (♦) corresponds exclusively to insulin in nanoprecipitated form. The condition with prior dilution (▪) corresponds to a mixture of nanoprecipitated insulin and non-precipitated insulin in solution.

FIG. 12. Size distribution of insulin nanoprecipitates obtained by dynamic light scattering.

FIG. 13. Evolution of glycemia in rat following subcutaneous administration of liquid insulin (Humuline®, Eli Lilly) and of Humuline® nanoprecipitates at a dose of 5 IU/kg/d. In this case, the Humuline® nanoprecipitates are administered in a 1% hyaluronic acid gel.

FIG. 14a. Evolution of glycemia in rat following subcutaneous administration of liquid insulin (Humuline®, Eli Lilly) and of Humuline® nanoprecipitates at a dose of 5 IU/kg/d. In this case, the Humuline® nanoprecipitates are administered in a polymer solution (175 mg/ml PLGA-PEG-PLGA polymer, 20% Capmul®, triacetin).

FIG. 14b. Evolution of insulinemia in rat following subcutaneous administration of liquid insulin (Humuline®, Eli Lilly) and of Humuline® nanoprecipitates at a dose of 5 IU/kg/d. In this case, the Humuline® nanoprecipitates are administered in a polymer solution (175 mg/ml PLGA-PEG-PLGA polymer, 20% Capmul®, triacetin). FIGS. 14a and 14b are derived from data collected from the same animals within a single experiment.

FIG. 15. Evolution of glycemia in rat following subcutaneous administration of liquid insulin (Humuline®, Eli Lilly) and of Humuline® nanoprecipitates at a dose of 5 IU/kg/d. In this case, the Humuline® nanoprecipitates are administered in a 1% hyaluronic acid, 10% Pluronic F127 gel.

FIG. 16. Scanning electron microscopy image of lyophilized insulin nanoprecipitates.

FIG. 17. Profiles of interleukin-2 (molecular mass of 15 kDa) release from three matrices containing interleukin-2 nanoprecipitates prepared according to the invention. (rhombuses: 3% propylene glycol alginate, squares: 10% hyaluronic acid, triangles: 4% Carbopol).

The following examples aim at illustrating the present invention.

### EXAMPLES

### Example 1: Preparation of Insulin Nanoprecipitates

In a flask, a solution of 0.468 mg of human insulin (Umuline Rapide® from Lilly at 100 IU/ml) is contacted with a PEG 550 solution (an organic nonsolvent of insulin), The preparation obtained is mixed by gentle stirring then the flask is placed in an ice bath for 30 minutes at 4° C. The mixture is then centrifuged using a range of centrifugal force between 10,000 and 50,000 g. Finally, the nanoprecipitates are collected and the quantity of insulin is determined by HPLC.

The nanoprecipitation yield is expressed as a percentage in relation to the insulin mass introduced at the start.

To measure the influence of insulin quantity on nanoprecipitation yield, the method above is repeated with an insulin mass of 0.936 mg, 1.404 mg, 1.874 mg and 2.340 mg, and the yield is calculated for each nanoprecipitation.

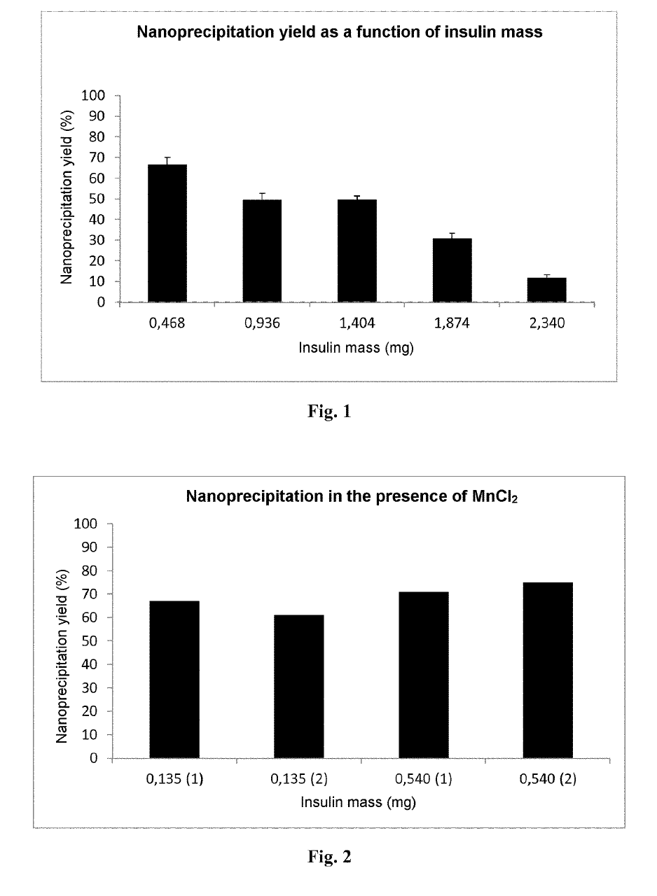

The results are shown in FIG. 1. It can thus be noted that the smaller the quantity of insulin to precipitate, the higher the nanoprecipitation yield.

The mean diameter of the nanoprecipitates obtained is measured by dynamic light scattering and is illustrated in FIG. 12. It can be noted that this mean diameter is less than 200 nm.

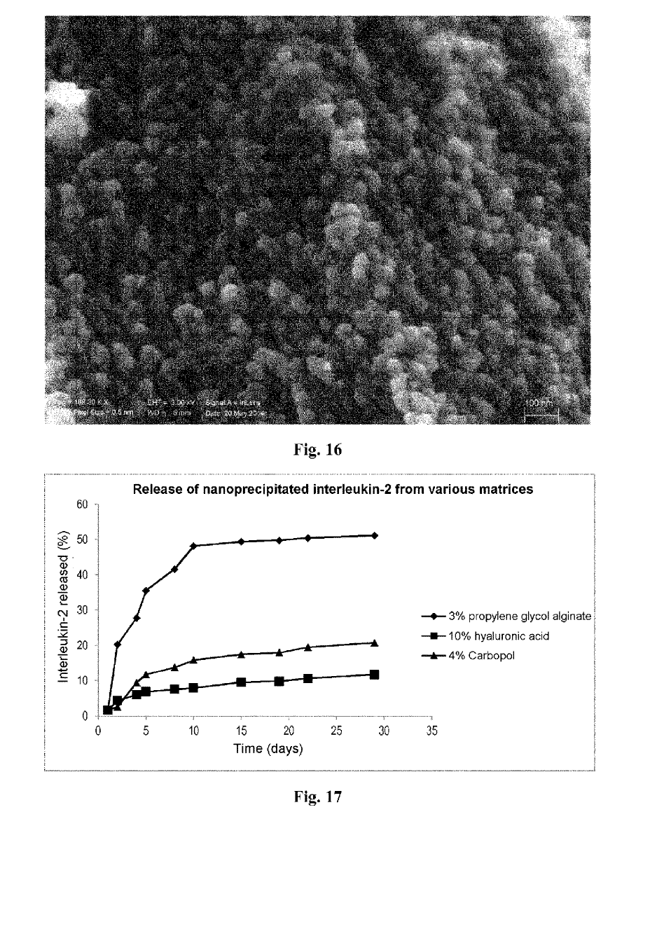

FIG. 16 is a scanning optical microscopy image of lyophilized insulin nanoprecipitates. The lyophilizate consists of all the insulin nanoprecipitates prepared according to the method described above, agglomerated to each other. It can be observed that each nanoprecipitate has a spherical shape and has a diameter between 50 and 100 nm. These sizes are close to those determined by the light scattering techniques.

### Example 2: Influence of the Presence of a Water-Soluble Salt During Nanoprecipitation Nanocoprecipitation of Insulin and MnCl₂, CaCl₂ or ZnCl₂

To measure the influence of the presence of a water-soluble salt on the yield of the insulin nanoprecipitation according to the invention, as a function of the quantity of salt and the quantity of insulin, three salts were studied: manganese chloride (MnCl₂), calcium chloride (CaCl₂) and zinc chloride (ZnCl₂). For each salt, four solutions to nanoprecipitate were prepared according to the quantities given in Table 1 below:

TABLE 1
Composition of solutions A, B C and D to nanoprecipitate

| | | | | |
|---|---|---|---|---|
| Solution | A | B | C | D |

| | | |
|---|---|---|
| Human insulin (mg) | 0.135 | 0.540 |

| | |
|---|---|
| PEG 550 (mg) | 510 |

| | | | | |
|---|---|---|---|---|
| Salt (mg) | 0.12 | 0.25 | 0.12 | 0.25 |

For each solution, human insulin (Umuline Rapide® from Eli Lilly at 100 IU/ml) is contacted with PEG 550 and the salt, in the quantities presented in Table 1. The preparation obtained is mixed by gentle stirring then the flask is placed in an ice bath for 30 minutes at 4° C. The mixture is then centrifuged using a range of centrifugal force between 10 000 and 50 000 g. Finally, the nanocoprecipitates are collected and the quantity of insulin is determined by HPLC.

The nanocoprecipitation yield is expressed as a percentage in relation to the quantity of insulin introduced at the start.

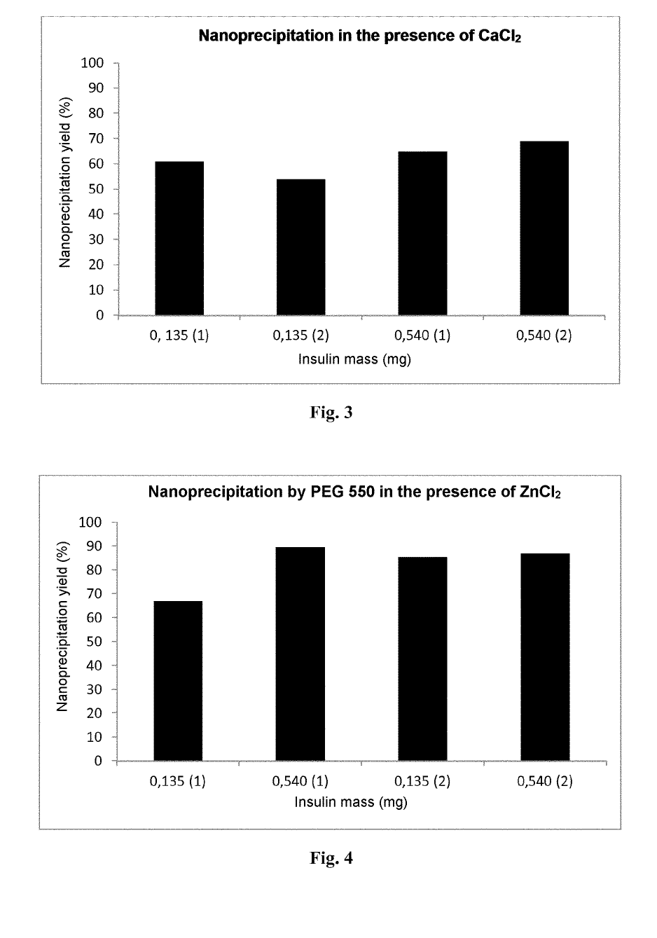

The results are shown in FIGS. 2, 3 and 4:
from FIGS. 2 and 3, it can be seen that the use of MnCl₂ or CaCl₂ makes it possible to obtain insulin nanoprecipitates with a yield close to 60-70% irrespective of the quantity of MnCl₂, CaCl₂ or insulin used;
from FIG. 4, it can be seen that the use of ZnCl₂ makes it possible to obtain insulin nanoprecipitates with a yield close to 80-90% irrespective of the quantity of MnCl₂ or insulin used.

The use of MnCl₂, CaCl₂ or ZnCl₂ thus has a dual advantage:
maximization of the nanoprecipitation yield,
formation of nanocoprecipitates having properties different from the nanoprecipitates prepared without salts.

In a general way, FIGS. 2, 3 and 4 illustrate the flexibility of the method according to the invention with respect to the nature of the salt used. Moreover, the same method (identical insulin mass, identical nonsolvent volume and nature) makes it possible to obtain different products in terms of physicochemical nature and biochemical properties by changing only the salt.

Nanocoprecipitation of Insulin and ZnCl₂

To supplement the results obtained in Example 2, new solutions are prepared according to the method of Example 2, in the quantities given in Table 2 below:

TABLE 2
Composition of solutions E, F, G, H, I, J, K, L, M, N, O, P to nanoprecipitate

| | Solution | | | | | | | | | | | |
|---|---|---|---|---|---|---|---|---|---|---|---|---|
| | E | F | G | H | I | J | K | L | M | N | O | P |
| Insulin (mg) | 0.936 | | | | 1.104 | | | | 1.872 | | | |
| PEG 550 (mg) | 510 | | | | | | | | | | | |
| ZnCl₂ (mg) | 0 | 0.12 | 0.18 | 0.25 | 0 | 0.12 | 0.18 | 0.25 | 0 | 0.12 | 0.18 | 0.25 |

The nanocoprecipitation yield is expressed as a percentage in relation to the insulin mass introduced at the start.

The results are shown in FIG. 5. From these results, it is to be noted that the highest yields are obtained for the largest masses of insulin. The effect of zinc chloride concentration is thus negligible.

We can thus conclude that:
zinc chloride makes it possible to optimize the nanoprecipitation yield, the yields obtained being close to 100%;
zinc chloride makes it possible to form nanocoprecipitates having specific properties.

### Example 3: Influence of Temperature on Nanoprecipitation or Nanocoprecipitation Yield

To measure the influence of temperature on the yield of the insulin nanoprecipitation according to the invention in the presence or absence of a water-soluble salt, the nanoprecipitation yields of two different solutions were studied for two different temperature conditions: at 4° C. and at room temperature. The first solution is prepared according to the method described in Example 1 and comprises 1.404 mg of human insulin (Umuline Rapide® from Lilly at 100 IU/ml). The second solution is prepared according to the method described in Example 2 and comprises 1.874 mg of human insulin (Umuline rapide@ from Lilly at 100 IU/ml) and 0.12 mg of zinc chloride.

The nanoprecipitation yield is expressed as a percentage in relation to the insulin mass introduced at the start.

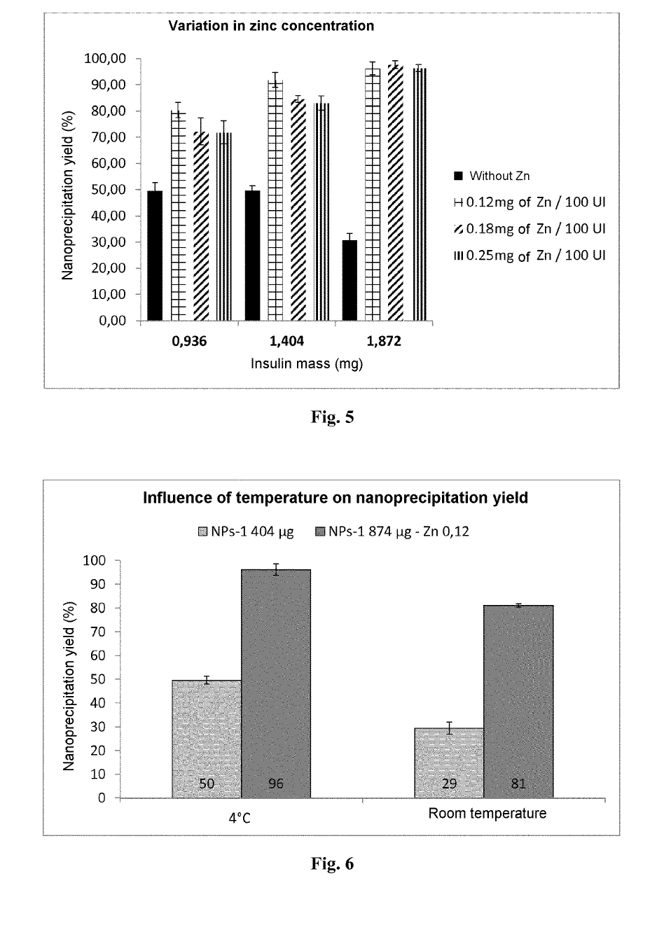

The results are shown in FIG. 6. They illustrate the fact that the nanoprecipitation can be carried out at room temperature, which introduces two important concepts:
flexibility of the method according to the invention and thus ease of use and of scaling;
notable difference with the methods described in the literature where temperature is a critical factor.

### Example 4: Profile of Insulin Release from Insulin Nanoprecipitates or Nanocoprecipitates According to the Invention from a Hyaluronic Acid Gel

To study the profile of insulin release from insulin nanoprecipitates according to the invention formulated within a polymer matrix, as well as the influence of the nature of the insulin nonsolvent, the insulin nanoprecipitates obtained according to the method described in Example 1, using PEG 550 or glycofurol as insulin nonsolvent, is formulated within a 1% hyaluronic acid gel.

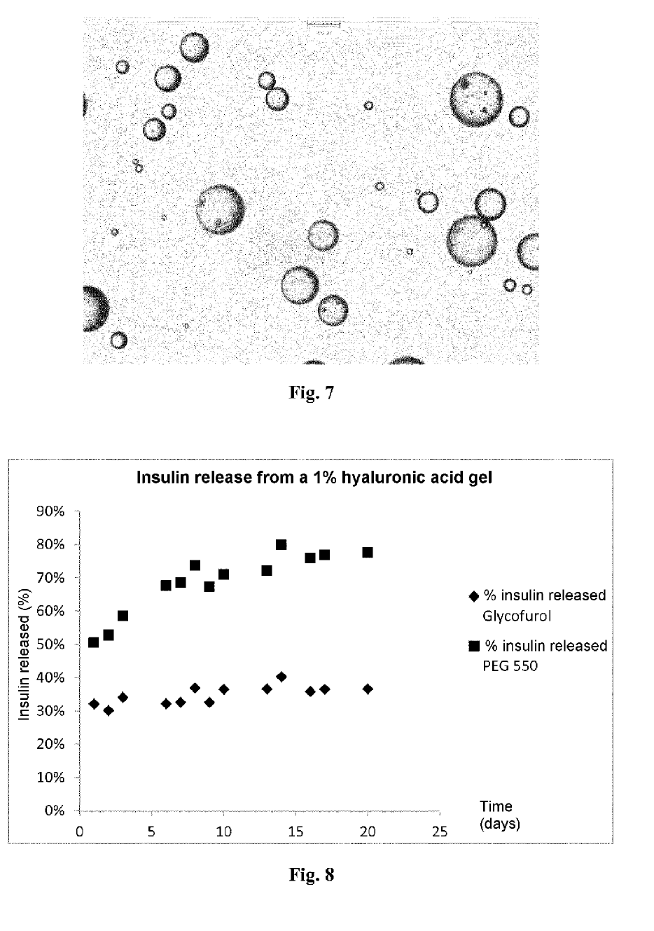

FIG. 7 shows the microspheres comprising the insulin nanoprecipitates (optical microscopy).

FIG. 8 shows the release of insulin from said insulin nanoprecipitates formulated within the 1% hyaluronic acid gel matrix. These results show that
the release occurs over more than 20 days, and
the nature of the nonsolvent influences the rate of release; the use of PEG 550 as insulin nonsolvent makes it possible to obtain a plasma insulin concentration close to 70% over more than 20 days, thus higher than when glycofurol is used (plasma concentration close to 30-40% over more than 20 days).

FIG. 9 shows the release of insulin from said insulin nanoprecipitates formulated within the 2% hyaluronic acid gel matrix.

Likewise, to study the profile of insulin release from a nanocoprecipitate of insulin and water-soluble salt according to the invention formulated within a polymer matrix, as well as the influence of the nature of the insulin nonsolvent and of the water-soluble salt, the insulin nanocoprecipitates obtained according to the method described in Example 2, using PEG 550 or glycofurol as insulin nonsolvent, and ZnCl₂ or MnCl₂ as water-soluble salt, are formulated within a 1% hyaluronic acid gel.

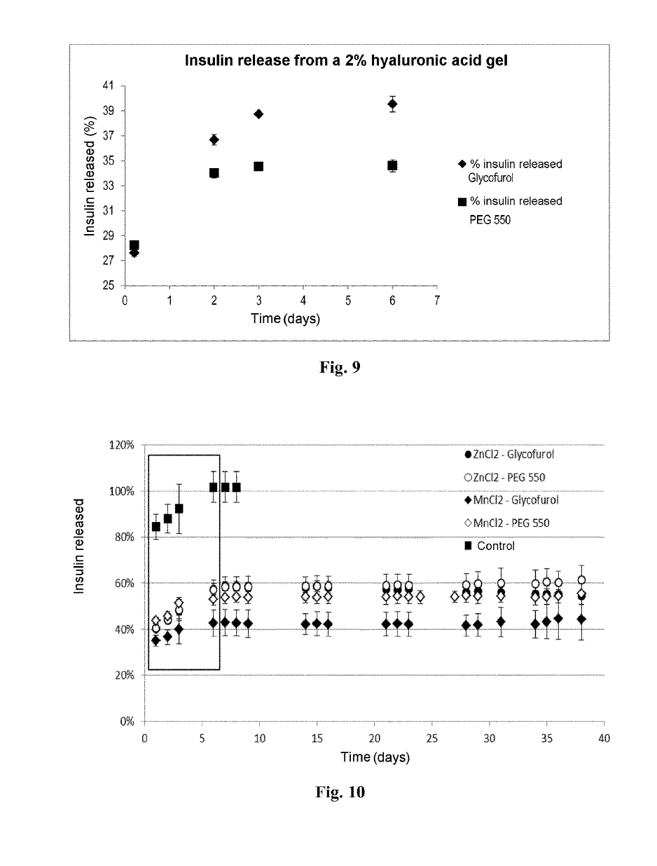

The profiles of insulin release from said nanocoprecipitates of insulin and water-soluble salt formulated within the 1% hyaluronic acid gel matrix are observed in FIG. 10 in comparison with the release profile of insulin in native form. These results show that
the release of insulin from the nanocoprecipitates occurs over more than 20 days whereas the release of insulin in native form is less than 1 day;
the nature of the nonsolvent influences the rate of release. The use of PEG 550 makes it possible to obtain a higher plasma insulin concentration than when glycofurol is used; and
the nature of the water-soluble salt also influences the rate of release. The use of ZnCl₂ makes it possible to obtain a higher plasma insulin concentration than when MnCl₂ is used.

### Example 5: Insulin Release Profile as a Function of the Presence or Absence of a Step of Re-Suspension of the Nanocoprecipitates According to the Invention

This study is carried out to determine the advantage of the reversibility of the nanoprecipitation or nanocoprecipitation method according to the invention. Indeed, when the nanoprecipitates or nanocoprecipitates according to the invention are suspended in solution, they regain their native, i.e. non-precipitated, form.

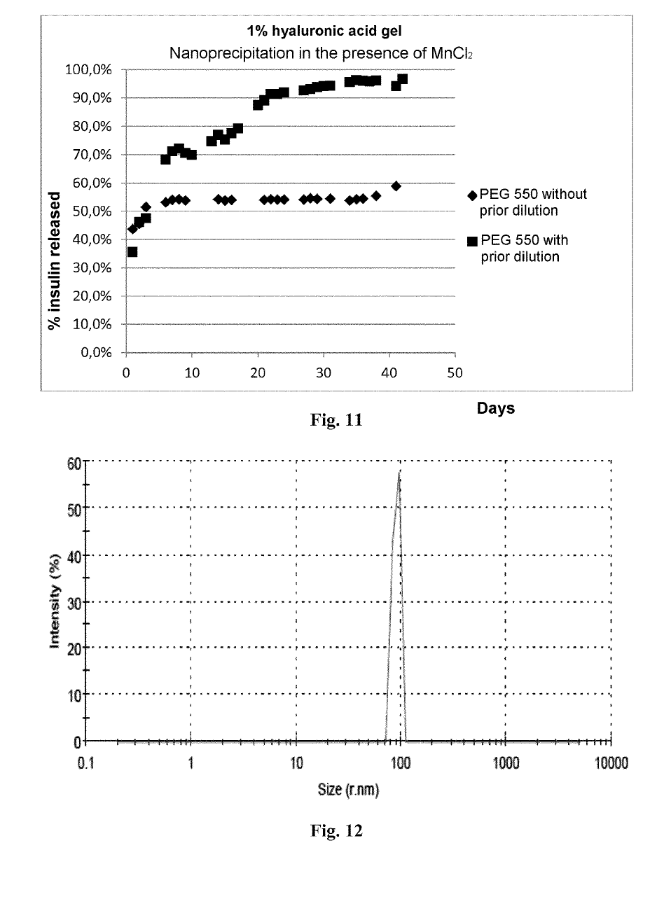

For this study, the profile of insulin release from nanocoprecipitates of insulin and MnCl₂ according to the invention, formulated in a hyaluronic acid gel matrix (condition without dilution), is compared with the release profile of these same nanocoprecipitates suspended in solution before being formulated in a hyaluronic acid gel matrix (condition with dilution). The results are shown in FIG. 11.

The reversibility of the nanoprecipitation or nanocoprecipitation according to the invention is a crucial process for several key reasons:
the biological activity of the protein or peptide is retained;
the ability of the nanoprecipitates to be an in vivo source of biotherapeutics from depot forms;
the ability to modulate the rate of release of the protein or peptide by playing with this reversibility (nanoprecipitates, partially suspended nanoprecipitates, peptide or protein free in solution).

### Example 6: Study of the Evolution of Glycemia and of Insulinemia in Rat Following the Administration of Insulin Nanoprecipitates

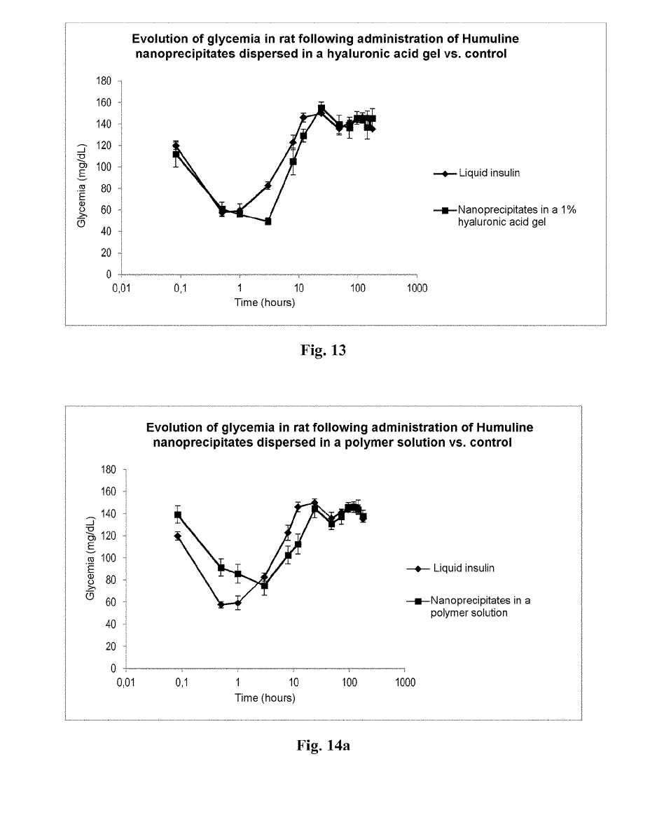

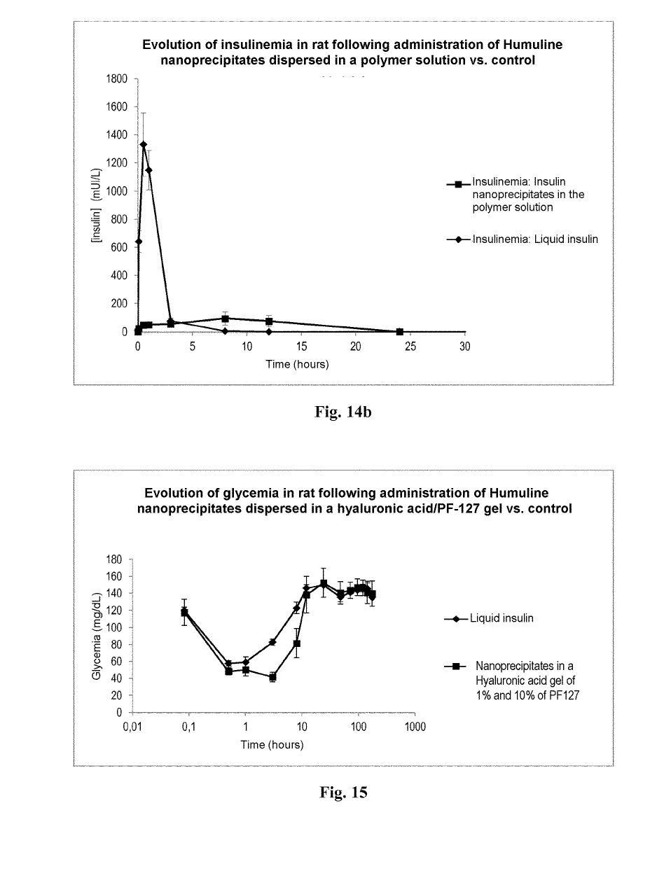

Three groups of six rats catheterized in the femoral vein were injected subcutaneously with the formulations tested at a dose of 5 IU/kg/d:
insulin (Humuline®) nanoprecipitates as prepared in Example 1 in a 1% hyaluronic acid gel (FIG. 13);
insulin (Humuline®) nanoprecipitates as prepared in Example 1 in a polymer solution (175 mg/ml PLGA-PEG-PLGA polymer, 20% Capmul®, triacetin) (FIGS. 14a and 14b); and
insulin (Humuline®) nanoprecipitates as prepared in Example 1 in a 1% hyaluronic acid, 10% Pluronic F127 gel (FIG. 15).

A control group of six rats catheterized in the femoral vein was injected subcutaneously with commercial Umuline® (Eli Lilly) solution at 5 IU/kg.

Blood samples were taken at precise times before and after injection of the test and control systems according to following schedule:
Three days before injection of the systems tested, basal glycemia was determined for all the rats of the study.
Six minutes before injection, blood samples were taken and designated as being the samples at T=0 minute.
After injection of the systems tested, blood samples were taken at the following times: 5 minutes, 30 minutes, 1 hour, 3 hours, 8 hours, 12 hours, 24 hours, 48 hours, 72 hours and 96 hours.

The glycemias were determined using the Accu-Chek Active® system from Roche. The insulinemias were determined using an ELISA kit marketed by Mercodia.

FIGS. 13 to 15 show the evolution of the glycemia or the insulinemia in rat following subcutaneous administration of liquid insulin (Humuline®, Eli Lilly) and of nanoprecipitates as prepared above.

The experiments carried out make it possible to compare the biological action of the insulin contained in the various formulations. The commercial liquid insulin formulation has a rapid action on glycemia between 30 and 60 minutes and presents a risk of causing hypoglycemias. Indeed, the pharmacokinetic profile has a peak activity at 30 minutes. Most of the insulin dose is delivered between 5 minutes and 1 hour after injection, which greatly increases the risk of hypoglycemia. The formulations based on insulin nanoprecipitates have a slower action on glycemia and make it possible, in certain cases, to limit the risks of the occurrence of hypoglycemias. Such is the case, for example, of the formulation based on insulin nanoprecipitates presented in FIG. 14a: the action of insulin is slowed down and the glycemia is controlled better because most of the insulin dose is delivered between 5 minutes and 12 hours.

The quantitative parameters for the formulation of Umuline® nanoprecipitates within a polymer carrier in comparison with the commercial Umuline® formulation (FIGS. 14a and 14b) are as follows:

| | Duration of | Minimum | Time corresponding |
|---|---|---|---|
| | action in | glycemia in | to the minimum |
| Formulation | hours | mg/dL | glycemia in hours |
| Liquid insulin | 8-12 | 58 | 0.5 |
| (Umuline ®) | | | |
| Humuline ® | 24 | 75 | 3 |
| nanoprecipitates in a | | | |
| polymer carrier | | | |

Concerning the evolution of the insulinemia (FIG. 14b), the use of a formulation based on insulin nanoprecipitates makes it possible to smooth the pharmacokinetic profile and to increase the plasma circulation time of the insulin by a factor of 3.

### Example 7: Preparation of Interleukin-2 Nanoprecipitates

Interleukin-2 (PROLEUKIN®—Novartis) is precipitated by addition of glycofurol (CAS 3169-2-85-0). For each test, a fixed volume of 1 ml of glycofurol is used; only the volume of the interleukin-2 solution varies. The solutions are left on ice for 30 minutes then centrifuged for 30 minutes at 21,382 G at 4° C. Nanoprecipitation tests are carried out for quantities of interleukin-2 ranging from 49 μg to 297 μg.

The supernatants are removed by aspiration then the interleukin-2 nanoprecipitates are taken up in 100 μl of Milli-Q water, and a 100 μl volume is assayed by addition of 5 ml of Bradford reagent. The samples are left in the dark for 5 minutes then absorbance is measured at 595 nm. The standard range was prepared over an interleukin-2 concentration range of 5 to 20 μg/ml, R²=0.99). The results of the assays are presented in Table 3 below:

TABLE 3
Nanoprecipitation yield

| Volume of interleukin-2 | Volume of glycofurol | Nanoprecipitation |
|---|---|---|
| solution (1.1 mg/ml) in μl | in ml | yield in % |
| 45 | 1 | 77 |
| 90 | 1 | 72 |
| 135 | 1 | 93 |
| 180 | 1 | 76 |

| | |
|---|---|
| Mean yield in % | 80 |

The nanoprecipitation yield is expressed as a percentage in relation to the mass of interleukin-2 introduced at the start.

The mean diameter of the nanoprecipitates obtained is measured using a Nanosizer Z from Malvern. This mean diameter is 14 nm.

### Example 8: Study of Interleukin-2 Release from Gel Matrices Containing Interleukin-2 Nanoprecipitates

The release experiments are carried out at 37° C. 198 μg of interleukin-2 nanoprecipitates, prepared according to the method described in Example 7, is mixed with 200 μl of gel matrix (10% hyaluronic acid, 3% propylene glycol alginate or 4% Carbopol) then placed in 1.5 ml polypropylene tubes. 1 ml of sterile DPBS (Dulbecco's Phosphate Buffered Saline, pH 7.4) supplemented with mannitol (1 g/l) and SDS (sodium dodecyl sulfate, 150 mg/1) is then added. Samples (1 ml) are taken at regular time intervals over a period of 30 days; after each sample is taken fresh medium is added to maintain a constant total volume. The assays are carried out using a commercially available ELISA kit (Thermo Scientific® Human IL-2 ELISA Kit).

FIG. 17 shows the release of interleukin-2 (molecular mass of 15 kDa) from three matrices containing interleukin-2 nanoprecipitates prepared according to the invention. These results show that the release occurs over more than 30 days.

This figure demonstrates that the formulations based on interleukin-2 nanoprecipitates make it possible to obtain sustained-releases of interleukin-2 in vitro. As a function of the type of polymer employed, the rates of release can be more or less rapid. In this example, the formulations based on hyaluronic acid and Carbopol make it possible to obtain continuous releases of interleukin-2 with no “burst” effect for durations equal to 30 days.

1. Method of non-denaturing preparation of peptide or protein nanoprecipitates or of peptide or protein and metal ion nanocoprecipitates, having a mean diameter of less than 1 μm, comprising the following steps:
a) preparation of a mixture of an aqueous solution of peptides or proteins, a nonsolvent of the peptides or proteins, and optionally a water-soluble metal salt;
b) gentle stirring of the mixture obtained in step a);
c) solid-liquid separation of the mixture obtained in step b); and
d) optionally, collection of the peptide or protein nanoprecipitates or the peptide or protein and metal ion nanocoprecipitates,
wherein said peptides or proteins have a molecular weight of 20 kDa or less, preferably of kDa or less, advantageously of 10 kDa or less, more advantageously of 8 kDa or less,
and said nonsolvent is selected from polyethylene glycols or polyethylene glycol derivatives having a molecular weight of less than 2,000 Da, advantageously between 200 and 2,000 Da, more advantageously of 550 Da, and organic diols selected from the group of hexylene glycol, butane-1,4-diol, pentane-1,5-diol, ethohexadiol, 2-methylpentane-2,4-diol(hexylene glycol), 3-cyclopentene-1,2-diol, cis-4-cyclopentene-1,3-diol, trans-1,4-dioxane-2,3-diol, 1,3-dioxane-5,5-dimethanol, (3S,4S)-pyrrolidine-3,4-diol, (3R,4R)-(−)-1-benzyl-3,4-pyrrolidinediol, (3S,4S)-(+)-1-benzyl-3,4-pyrrolidinediol, 3-cyclopentene-1,2-diol, 2-methyl-butane-1,3-diol.

2. Method according to claim 1, characterized in that the mean diameter of the nanoprecipitates or nanocoprecipitates is between 5 nm and 500 nm, particularly between 5 and 200 nm, more particularly between 5 and 170 nm.

3. Method according to claim 1, characterized in that said metal on is selected from Zn, Mg, Ca, Mn, Fe, U or Cu ions, advantageously said metal ion is Zn or Mn.

4. Method according to claim 1, characterized in that said nonsolvent is selected from PEG 550, glycofurol or hexylene glycol, particularly from PEG 550 and hexylene glycol, even more particularly said nonsolvent is PEG 550.

5. Method according to claim 1, characterized in that said peptides or proteins are therapeutic.

6. Method according to claim 1, characterized in that said peptides or proteins are selected from human insulin, growth hormone, glucagon, peptide hormones or a therapeutically effective derivative or fragment thereof.

7. Method according to claim 1, characterized in that said water-soluble metal salt is selected from ZnCl2, MgCl2, CaCl2, MnCl2, FeCl2, LiCl and CuSO4, advantageously ZnCl2 or MnCl2.

8. Method according to claim 1, characterized in that said solid/liquid separation of step c) is a tangential filtration or a centrifugation.

9. Peptide or protein nanoprecipitates or peptide or protein and metal ion nanocoprecipitates, obtainable by the preparation method according to claim 1.

10. Nanoprecipitates or nanocoprecipitates according to claim 9, characterized in that the peptide or the protein is human insulin or a derivative or a therapeutically effective fragment.

11. Nanoprecipitates or nanocoprecipitates according to claim 9, for use as a medicinal product.

12. Nanoprecipitates or nanocoprecipitates according to claim 9, for use as a medicinal product for single parenteral administration such that the duration during which the plasma concentration of said peptides or proteins is within the therapeutic window is greater than the duration during which the plasma concentration after a single parenteral administration of the same peptides or proteins not prepared according to the method of preparation as claimed in claims 1 to 8 is within the therapeutic window.

13. Sustained-release pharmaceutical composition comprising nanoprecipitates or nanocoprecipitates according to claim 9.

14. Sustained-release pharmaceutical composition according to claim 13, for use as a medicinal product.

15. Pharmaceutical composition according to claim 13, characterized in that the peptide or the protein is selected from human insulin, growth hormone, glucagon, peptide hormones or a therapeutically effective derivative or fragment thereof.
